(19) United States
(12) Patent Application Publication
(10) Pub. No.: US 2023/0267616 A1
(43) Pub. Date: Aug. 24, 2023

(54) IMAGE SEGMENTATION SYSTEM AND METHOD

(71) Applicants: Singapore Health Services PTE LTD, Singapore (SG); Nanyang Technological University, Singapore (SG)

(72) Inventors: Dheo Arokhim Yusufi CAHYO, Singapore (SG); Han Nian Marcus ANG, Singapore (SG); Leopold SCHMETTERER, Singapore (SG); Ai Ping YOW, Singapore (SG); Wing Kee Damon WONG, Singapore (SG)

(21) Appl. No.: 18/043,552

(22) PCT Filed: Sep. 2, 2021
(86) PCT No.: PCT/SG2021/050530

§ 371 Date: Feb. 28, 2023

(30) Foreign Application Priority Data
Sep. 2, 2020 (SG) 10202008522X

(51) Int. Cl. G06T 7/11; G06T 7/174; G06T 3/40; G06T 7/00; G06T 11/00; A61B 3/10; A61B 3/12

(52) U.S. Cl. CPC G06T 7/11; A61B 3/102; A61B 3/1225; G06T 3/40; G06T 7/0012; G06T 7/174; G06T 11/003; G06T 2207/10101; G06T 2207/20084; G06T 2207/30041

(57) ABSTRACT

Disclosed herein is a method of segmenting a volumetric image comprising a plurality of slices, the method comprising: inputting a target slice of the volumetric image to a deep neural network (DNN) having a multi-task learning architecture, the multi-task learning architecture comprising: a segmentation DNN that is configured to output a segmentation of the target slice; and a reconstruction DNN that is configured to: receive a plurality of adjacent slices to the target slice; and output a reconstruction of the target slice based on the plurality of adjacent slices; wherein the reconstruction DNN is further configured to share spatial information with the segmentation DNN, the spatial information being indicative of correlations between the adjacent slices and the target slice.

### TECHNICAL FIELD

The present disclosure relates, in general terms, to methods and systems for image segmentation. It is particularly, though not exclusively, applicable to segmentation of medical images, such as volumetric image data from optical coherence tomography.

### BACKGROUND

An essential task in the clinical use of medical images is segmentation, which is the task of detecting, identifying and subsequently quantifying regions of interest in an acquired scan for signs of pathology. Segmentation is important as it allows the quantitative monitoring of biomarkers, such as tissue characteristics, for detection and monitoring of abnormalities, which has applications in disease screening and management.

Various methods have been reported for medical image segmentation. A two-dimensional approach for medical image segmentation has successfully been demonstrated in different applications. Many architectures for volumetric segmentation have been developed based on the U-Net architecture. A similar approach was also proposed in developing V-Net. However, these approaches usually require the whole volumetric image to be considered, which is potentially computationally expensive with extensive memory requirements.

Recently, recurrent networks have been gaining popularity due to their sequential approach for volumetric medical images. One known application of recurrent networks uses a combination of Fully Convolutional Networks (FCN) and Recurrent Neural Networks (RNN). However, this still requires the whole medical scan to be available. Addressing this issue, several approaches to combine the sequential property of spatial context in medical images with two-dimensional segmentation have been proposed. However, these methods are computationally heavy and are prone to memory leakages which are common in recurrent networks.

A network called D-UNet enables the architecture to learn spatial context of adjacent slices during the encoding stage using three-dimensional convolution and by combining it with two-dimensional segmentation that treated the adjacent slices as different channels. One way is to learn the spatial context in the same manner using a proposed Globally Guided Progressive Fusion Network (GGPF-Net). These methods successfully outperformed and replaced sequential and three-dimensional FCN approaches in terms of performance and computation efficiency. However, the spatial context learnt by such architectures is dependent on the quality of the labels available.

It would be desirable to overcome or alleviate at least one of the above-described problems, or at least to provide a useful alternative.

### SUMMARY

Disclosed herein is a method of segmenting a volumetric image comprising a plurality of slices, the method comprising:
inputting a target slice of the volumetric image to a deep neural network (DNN) having a multi-task learning architecture, the multi-task learning architecture comprising:
a segmentation DNN that is configured to output a segmentation of the target slice; and
a reconstruction DNN that is configured to:
   receive a plurality of adjacent slices to the target slice; and
   output a reconstruction of the target slice based on the plurality of adjacent slices;
wherein the reconstruction DNN is further configured to share spatial information with the segmentation DNN, the spatial information being indicative of correlations between the adjacent slices and the target slice.

In some embodiments, the reconstruction DNN comprises a convolutional feature extractor for generating first feature data from the adjacent slices, and a reconstruction downsampler for generating first reduced-dimension feature data from the first feature data at one or more scales.

In some embodiments, the reconstruction DNN comprises a reconstruction upsampler for transforming the first reduced-dimension feature data to first upsampled data having the same dimensions as the first feature data.

In some embodiments, the reconstruction DNN comprises one or more dimension reduction layers for applying a dimension reduction mechanism to the first feature data and/or to the first reduced-dimension feature data.

In some embodiments, the dimension reduction mechanism comprises:
inputting the first feature data and/or the first reduced-dimension feature data to a 3D convolution layer;
applying an aggregation of features between adjacent slices;
applying batch normalization to the output of the 3D convolution layer; and
applying a ReLU activation function to the output of the batch normalization.

In some embodiments, layers of the reconstruction downsampler are connected to layers of the reconstruction upsampler via respective ones of the dimension reduction layers by concatenation.

In some embodiments, the segmentation DNN comprises a convolutional feature extractor for generating second feature data from the target slice, and a segmentation downsampler for generating second reduced-dimension feature data from the second feature data at one or more scales.

In some embodiments, the segmentation DNN comprises a segmentation upsampler for transforming the second reduced-dimension feature data to second upsampled data having the same dimensions as the second feature data.

In some embodiments, layers of the segmentation downsampler are connected to layers of the segmentation upsampler.

In some embodiments, the reconstruction DNN is configured to share spatial information with the segmentation DNN by element-wise addition of output of layers of the reconstruction upsampler to output of layers of the segmentation upsampler.

In some embodiments, the loss function of the segmentation DNN is the 2D Intersection over Union (IoU) loss function.

In some embodiments, the volumetric image is a 3D medical image.

In some embodiments, the 3D medical image is a 3D optical coherence tomography (OCT) image.

In some embodiments, the 3D OCT image is a retinal image, and wherein the target slice corresponds to a layer of the choroid.

In some embodiments, the method is repeated for a plurality of target slices, and wherein the method further comprises generating a choroidal thickness map from segmentation of the plurality of target slices.

Also disclosed herein is a system for segmentation of a volumetric image comprising a plurality of slices, the system comprising:
at least one processor; and
computer-readable storage having stored thereon instructions for causing the at least one processor to carry out the disclosed method.

Further disclosed herein is a non-transitory computer-readable storage having instructions stored thereon for causing at least one processor to carry out the disclosed method.

### BRIEF DESCRIPTION OF THE DRAWINGS

Embodiments will now be described, by way of non-limiting example, with reference to the drawings in which:

FIG. 1 is a flow diagram showing a method used for segmenting a volumetric image comprising a plurality of slices;

FIG. 2 is a flow diagram showing another method used for segmenting a volumetric image comprising a plurality of slices;

FIG. 3 is a flow diagram showing a dimension reduction mechanism;

FIG. 4 is a flow diagram showing another method used for segmenting a volumetric image comprising a plurality of slices;

FIG. 5 is a neural network architecture diagram showing a spatial aggregated network consisting of reconstruction and segmentation branches;

FIG. 6 illustrates respective two-dimensional segmentations generated by a method consistent with the present disclosure, and by various prior art methods;

FIG. 7 illustrates a choroidal thickness map generated from the ground truth and from each of the segmentation architectures used in FIG. 6; and

FIG. 8 is a schematic diagram showing components of an exemplary computer system for performing the methods described herein.

### DETAILED DESCRIPTION

The present disclosure relates to a computationally efficient and accurate segmentation approach, which is robust to interstitial variations, for the segmentation of volumetric medical images. In the present disclosure, a novel segmentation multi-task learning architecture that is capable of fully automated three-dimensional segmentation of volumetric medical image data is proposed for volumetric segmentation. The proposed architecture incorporates both reconstruction and segmentation tasks. Simultaneous reconstruction and segmentation extracts intra-slice features which are directly used for segmentation. In particular, the multi-task learning architecture aggregates the spatial context in adjacent cross-sectional slices to reconstruct a central slice. Said multi-task learning architecture reconstructs said central slice by learning the spatial information between the adjacent slices. Soft parameter sharing between the reconstruction and segmentation tasks may be used to channel the spatial information. Said soft parameter sharing aggregates the spatial features more explicitly by directly learning the correlation between adjacent slices and the slices that will be segmented.

Spatial context learnt by the proposed reconstruction mechanism may be fused using a U-Net-based architecture. In the present disclosure, the proposed U-Net-based architecture is referred to as Spatial Aggregated Networks (SA-Net) due to its aggregation of spatial information. SA-Net learns the spatial information between adjacent cross-sections to reconstruct a selected cross-section. Said SA-Net is a convolutional neural network that is based on a fully convolutional network and its architecture can be modified and extended to work with fewer training images and to yield more precise segmentations. The main idea of the proposed U-Net-based architecture is to supplement a contracting network by successive layers, where pooling operations are replaced by upsampling operators. Hence these layers increase the resolution of the output. Further, a successive convolutional layer can then learn to assemble a precise output based on this information. In the proposed SA-Net, there are a large number of feature channels in the upsampling part, which allows the network to propagate context information to higher resolution layers. It will be appreciated that incorporating spatial information from corresponding adjacent slices enables the proposed SA-Net architecture to explicitly integrate spatial correspondences. In general, the present disclosure does not require the whole volumetric image to be considered, thus avoiding costly computation and extensive memory requirements. At the same time, the proposed approach is not computationally heavy and is also not prone to memory leakage problems which are common in recurrent networks.

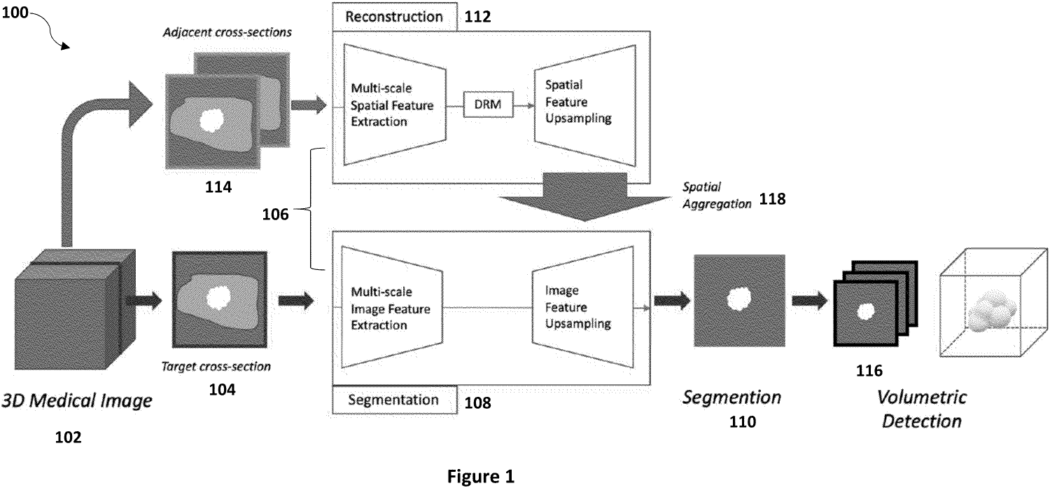

FIG. 1 illustrates an example method 100 of segmenting a volumetric image 102 comprising a plurality of slices. Broadly, the method 100 comprises:
inputting a target slice 104 of the volumetric image 102 to a deep neural network (DNN) (e.g., SA-Net) having a multi-task learning architecture 106, the multi-task learning architecture 106 comprising:
a segmentation DNN 108 that is configured to output a segmentation 110 of the target slice 104; and
a reconstruction DNN 112 that is configured to:
   receive a plurality of adjacent slices 114 to the target slice 104; and
   output a reconstruction 116 of the target slice 104 based on the plurality of adjacent slices 114;
wherein the reconstruction DNN 112 is further configured to share spatial information 118 with the segmentation DNN 108, the spatial information 118 being indicative of correlations between the adjacent slices 114 and the target slice 104.

As shown in FIG. 1, the volumetric image could be a three-dimensional (3D) volume which encompasses the target region and that is first acquired by a medical imaging system. The proposed SA-Net architecture 106 incorporates both reconstruction and segmentation tasks. The segmentation DNN 108 performs the segmentation, while spatial context is provided by the adjacent slices in the reconstruction DNN 112. In particular, each cross-sectional scan 114 to be segmented by the SA-Net in the segmentation branch is processed concurrently with its adjacent scans 114 in the reconstruction branch 112 which supplies the spatial context. In the present disclosure, specifically, given a slice Iᵢ to be segmented, the reconstruction branch will take Λ = {Iᵢ₋ₙ, ..., Iᵢ₋₁, Iᵢ₊₁, ..., Iᵢ₊ₙ} for its input where 2n defines the number of adjacent slices to be used.

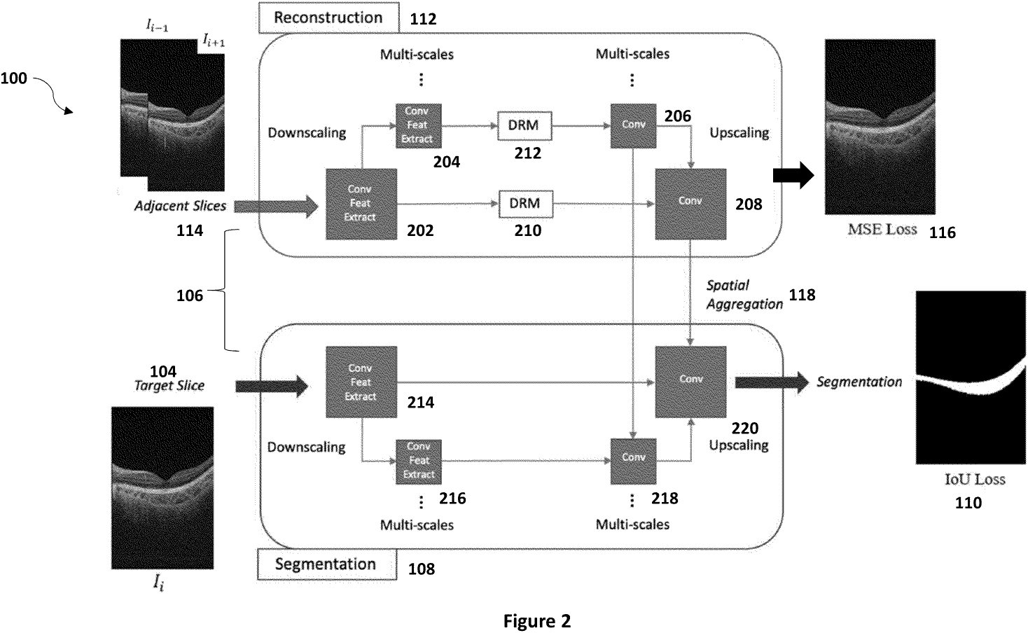

Detailed connections between the segmentation DNN 108 and reconstruction DNN 112 are shown in FIG. 2. The present disclosure will discuss the construction DNN 112 and segmentation DNN 108, respectively.

In the reconstruction DNN 112, explicit spatial information from the adjacent slices 114 may be extracted. In particular, explicit spatial information from the adjacent slices 114 may be extracted by using a series of 3D convolutions. It will be appreciated that the reconstruction DNN 112 can be divided into downsampling and upsampling parts. The adjacent slices 114 are downsampled and the convolutions are repeated to extract multi-scale representations of spatial context. In some embodiments, the reconstruction DNN 112 comprises a convolutional feature extractor 202 for generating first feature data from the adjacent slices 114. The adjacent slices 114 are then downsampled and the convolutions are repeated to extract multi-scale representations of spatial context. During the downsampling process, rich spatial information is exploited from the adjacent slices 114 by using 3D convolution and max pooling layers. In one embodiment as shown in FIG. 2, the reconstruction DNN 112 further comprises a reconstruction downsampler 204 for generating first reduced-dimension feature data from the first feature data at one or more scales. Said reconstruction downsampler 204 enables the architecture 106 to learn local spatial information contained between slices, that is, the inter-slice features.

After the downsampling stage, convolutional upsampling is later performed at different levels to ensure consistent representation of information from different scales, and is concatenated with the residuals at the same scale. In some embodiments, the reconstruction DNN 112 comprises a reconstruction upsampler 206 for transforming the first reduced-dimension feature data generated by the reconstruction downsampler 204 to first upsampled data having the same dimensions as the first feature data at one or more scales. After upsampling a final 2D convolution is performed and the loss between output and the ground truth (i.e., the Iᵢ slice) is calculated. Embodiments of the present disclosure use mean squared error to calculate the similarity distance between the predicted output y^{pred} and ground truth y^{true}.

$${L}_{\text{reconst}} = \frac{1}{M N} {\sum }_{i = 1}^{M} {\sum }_{i = 1}^{N} {\left[{y}_{i , j}^{\text{pred}} - {y}_{i , j}^{\text{true}}\right]}^{2}$$

Other similarity or dissimilarity measurement such as SSIM (structural similarity) index may also be used.

In some embodiments, the reconstruction DNN 112 may further comprise one or more dimension reduction layers for applying a dimension reduction mechanism (DRM) 210 to the first feature data generated by the convolutional feature extractor 202. The reconstruction DNN 112 may also comprise one or more dimension reduction layers for applying another DRM 212 to the first reduced-dimension feature data generated by the reconstruction downsampler 204. In the present disclosure, said DRM 210 and 212 are used for a more efficient representation of the information. In particular, to reduce the number of parameters given by the 3D convolution layers, in the bottleneck block the present disclosure incorporates said DRM 210 and 212 to convert 3D information into two-dimensional (2D) information. In some embodiments, the converted 3D information generated by the DRMs 212 and 210 are then upsampled using 2D convolution layers in reconstruction upsamplers 206 and 208, respectively.

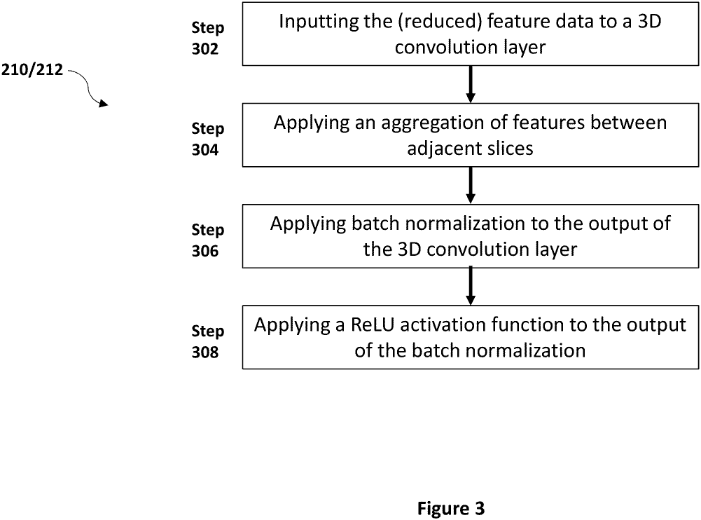

FIG. 3 shows an example workflow of the proposed DRM 210/212. In general, said DRM starts with a 3D convolution layer, followed by batch normalization and Rectified Linear Unit (ReLU) activation. In one embodiment, the DRM 210/212 comprises first inputting the first feature data generated by the convolutional feature extractor 202 and/or the first reduced-dimension feature data generated by the reconstruction downsampler 204 to a 3D convolution layer (step 302). The DRM 210/212 further comprises applying an aggregation of features between the adjacent slices 114 (step 304). In some embodiments, during step 304, the numerical features along the cross-sectional axis are summed. At step 306, batch normalization is applied to the output of the 3D convolution layer. Said batch normalization is a method used to make neural networks faster and more stable through normalization of the layers’ inputs by re-centering and re-scaling. Finally at step 308, a ReLU activation function is applied to the output of the batch normalization. In the context of artificial neural networks, said ReLU activation function is defined as the positive part of its argument. In some embodiments, other activation functions, such as Leaky ReLU, exponential linear unit (ELU), or Parametric Rectified Linear Unit (PReLU) may be used instead of ReLU. It will be appreciated that the proposed DRM ensures the features of the volumetric nature of adjacent slices are retained and at the same time scaled or normalized. Furthermore, it reduces the complexity of the reconstruction branch and also increases the converging speed.

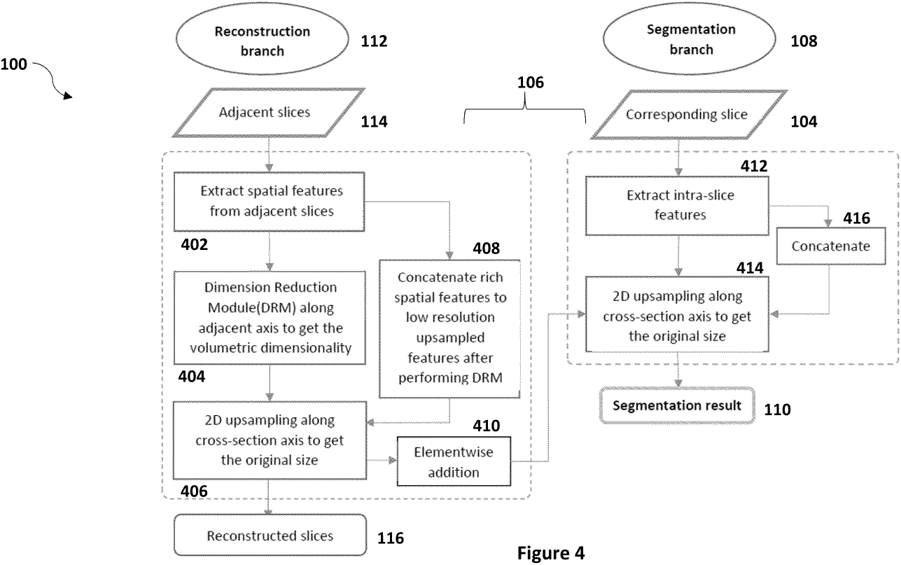

FIG. 4 illustrates another example of the proposed multi-task learning architecture 106 comprising the reconstruction DNN 112. Said reconstruction DNN 112 takes the adjacent slices as input and extracts spatial features. The dimension is reduced along the cross-section axis to get volumetric dimensionality. Upsampling is performed to get the original 3D size. As will be discussed in details, the spatial information (see step 410) from the reconstruction DNN 112 is fused to the segmentation DNN 108 that takes corresponding slices to be segmented as the input.

As shown in FIG. 4, the first step (step 402) is to extract spatial features from the adjacent slices 114. The reconstruction DNN 112 may comprise the convolutional feature extractor 202 for generating the first feature data from the adjacent slices 114. The reconstruction DNN 112 may further comprise the reconstruction downsampler 204 for generating the first reduced-dimension feature data from the first feature data at one or more scales. The reconstruction DNN 112 may comprise one or more dimension reduction layers for applying the DRM 210 to the first feature data. The reconstruction DNN 112 may also comprise one or more dimension reduction layers for applying another DRM 212 to the first reduced-dimension feature data generated by the reconstruction downsampler 204. In particular, at step 404, the DRM 210/212 along the adjacent axis is applied to get the volumetric dimensionality. For the DRM 210/212, the dimension along the cross-section axis. At step 406, the reconstruction DNN 112 comprises reconstruction upsamplers 206 and 208 for transforming the first reduced-dimension feature data generated by the reconstruction downsampler 204 to first upsampled data having the same dimensions as the first feature data at one or more scales. At step 406 said 2D upscaling along the cross-section axis is applied to get the original 3D size. The architecture 106 finally outputs the reconstruction 116 of the target slice 104 based on the plurality of adjacent slices 114.

In some embodiments, as illustrated in FIG. 4, layers of the reconstruction downsampler 204/208 are connected to layers of the reconstruction upsampler 206/208 respectively via respective ones of the dimension reduction layers by concatenation. To be more specific, after performing the DRM, the reconstruction DNN 112 may concatenate rich spatial features to low resolution upsampled features (see step 408). As a result of this architecture, the proposed method does not need to evaluate the complete volumetric image, which saves time and money in processing and memory.

In the segmentation DNN 108, explicit spatial information from the target slice 104 may be extracted (see FIGS. 1 and 2). Said target slice 104 as the input refers to the corresponding slice to be segmented. An approach similar to the one used in the reconstruction DNN 112 is used to extract information within the target slice at multiple scales. In particular, explicit spatial information from the target slice 104 may be extracted by using a series of 2D convolutions. It will be appreciated that the segmentation DNN 108 can be divided into downsampling and upsampling parts. The target slice 104 is downsampled and the convolutions are repeated to extract multi-scale representations of spatial context. The segmentation DNN 108 may comprise a convolutional feature extractor 214 for generating second feature data from the target slice 104. The target slice 104 is then downsampled and the convolutions are repeated to extract multi-scale representations of spatial context. During the downsampling process, intra-slice features contained within the target slice 104 is exploited from the target slice 104 by using 2D convolution and max pooling layers. In one embodiment as shown in FIG. 2, the segmentation DNN 108 further comprises a reconstruction downsampler 216 for generating second reduced-dimension feature data from the second feature data at one or more scales.

After the downsampling stage, convolutional upsampling is later performed at different levels to ensure consistent representation of information from different scales, and is concatenated with the residuals at the same scale. In some embodiments, the segmentation DNN 108 comprises segmentation upsamplers 218 and 220 for transforming the second reduced-dimension feature data to second upsampled data having the same dimensions as the second feature data. In the upsampling part, high-resolution features during downsampling are concatenated with low-resolution features. In each end of the upsampling block, consisting of one 2D upsampling layer and two 2D convolution layers, the knowledge of the inter-slice features from the reconstruction branch is fused. The high-resolution 2D volumetric features are added element-wise with 2D intra-slice extracted features to incorporate the inter-correlation features between slices.

As shown in FIG. 4, the segmentation DNN 108 takes the target slice 104 as input and extracts intra-slice features. Upsampling is performed to get the original 3D size. In some embodiments, layers of the segmentation downsampler are connected to layers of the segmentation upsampler. As shown in FIG. 2, the spatial information 118 from the reconstruction DNN 112 is fused to the segmentation DNN 108 that takes corresponding slices to be segmented as the input.

As shown in FIG. 4, the first step (step 412) is to extract intra-slice features from the target slice 104. The segmentation DNN 108 may comprise the convolutional feature extractor 214 for generating the second feature data from the target slice 104. The segmentation DNN 108 may further comprise the segmentation downsampler 214 for generating the first reduced-dimension feature data from the second feature data at one or more scales. At step 414, the reconstruction DNN 112 comprises segmentation upsamplers 218 and 220 for transforming the first reduced-dimension feature data generated by the segmentation downsampler 216 to first upsampled data having the same dimensions as the first feature data at one or more scales. At step 406 said 2D upscaling along the cross-section axis is applied to get the original 3D size. It will be appreciated that, the reconstruction DNN may be configured to share spatial information with the segmentation DNN by element-wise addition (see 410 in FIG. 4) of output of layers of the reconstruction upsampler to output of layers of the segmentation upsampler. The architecture 106 finally outputs the segmentation result 110.

In embodiments as illustrated in FIG. 4, layers of the segmentation downsampler 204/208 are connected to layers of the reconstruction upsampler 218/220 respectively via respective ones of the dimension reduction layers by concatenation. To be more specific, the segmentation DNN 112 may concatenate rich intra to low resolution upsampled features (see step 416). As a result of this architecture, the proposed method does not require consideration of the entire volumetric image, reducing costly processing and memory requirements.

The loss function of the segmentation DNN may be the 2D Intersection over Union (IoU) loss function. The upsampling DNN is ended with a one-by-one 2D convolution and a sigmoid activation function. The present disclosure uses 2D IoU loss function to maximize the intersection region between the prediction and the ground truth. This 2D IoU loss function is defined as

$${L}_{I o U} = 1 - \frac{{\sum }_{i , j ∈ ℕ} {y}_{i , j}^{\text{pred}} {y}_{i , j}^{\text{true}}}{{\sum }_{i , j ∈ ℕ} {y}_{i , j}^{\text{pred}} + {y}_{i , j}^{\text{true}} - {y}_{i , j}^{\text{pred}} {y}_{i , j}^{\text{true}}} .$$

In some embodiments, the volumetric image is a 3D medical image. In particular, the proposed SA-Net could potentially be applied for the segmentation and detection of structures in medical imaging modalities that acquire 3D volumetric data, which include but are not limited to Optical Coherence Tomography, Computed Tomography and Magnetic Resonance Imaging. The 3D medical image may be a 3D optical coherence tomography (OCT) image. Said OCT refers to a relatively recent medical imaging approach which enables high resolution depth-resolved imaging of structures below the surface of the retina. This allows visualization of sub-retinal changes which were not observable using fundus photography. The utility of OCT imaging has led to its widespread adoption in many clinical practices and has even replaced fundus photography as the main form of ophthalmic imaging for some practices. In the present disclosure, the 3D OCT image may be a retinal image, and the target slice may correspond to a layer of the choroid.

Also disclosed herein is a system for segmentation of a volumetric image comprising a plurality of slices, comprising at least one processor; and computer-readable storage having stored thereon instructions for causing the at least one processor to carry out the disclosed method.

### Experiment

After pre-processing, embodiments of the present disclosure use a five-fold cross-validation strategy to train and evaluate the proposed model. To avoid training bias and risk of overfitting, it is ensured that all images from the same eye were in the same fold. This avoids a scenario where the testing and training partitions could potentially consist of different images from the same eye. The overall experimental result is then obtained by averaging over all validation sets in each fold. The architecture is developed using Python version 3.7.4 and TensorFlow version 2.0. Experiments were conducted using a workstation with GPU NVIDIA RTX 2080 Ti and 64GB RAM.

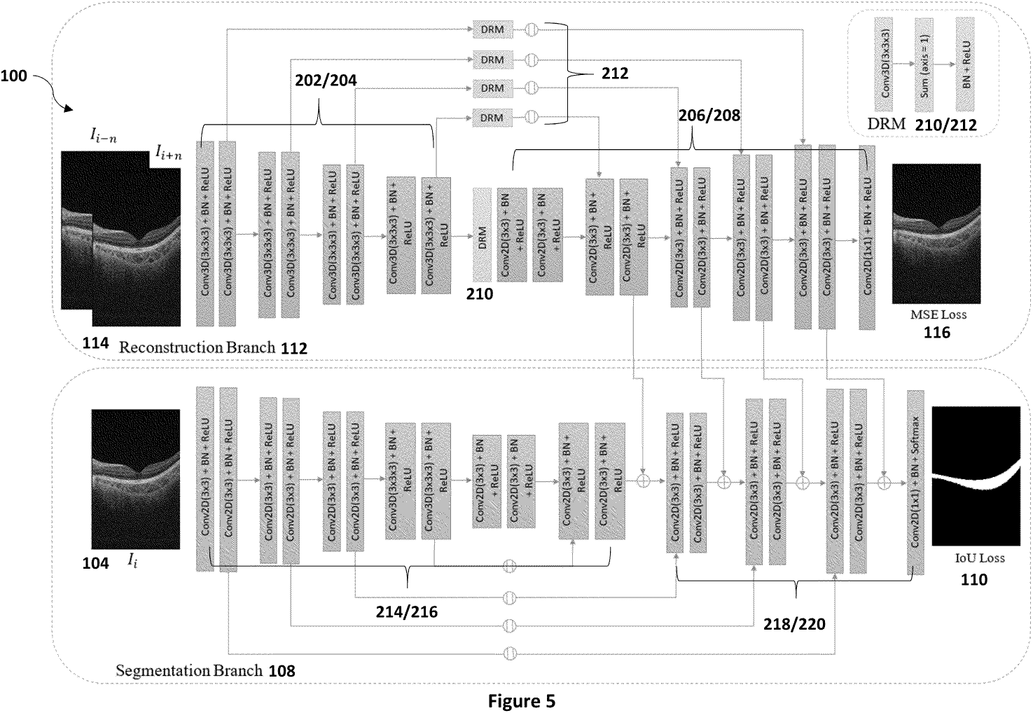

FIG. 5 illustrates the proposed SA-Net 100 consisting of the reconstruction and segmentation branches (i.e., 112 and 108) for two different tasks to learn inter-slice and intra-slice features respectively. In particular, FIG. 5 illustrates the implementation of the proposed SA-Net for a particular application (i.e., OCT imaging of the choroid or RPE). Spatial information aggregated in reconstruction branch 112 is fused to the segmentation branch 108 during the encoding stage. The SA-Net 100 extracts spatial features from adjacent slices by reconstructing the target slice. The implementation of the SA-Net 100 for choroid segmentation makes use of the volumetric nature of the OCT images, as well as the availability of the data set. OCT choroidal segmentation is highly challenging especially in high myopes and the results from SA-Net demonstrate the advantages of the approach. Segmentation of the choroid is important not just for myopia but also other conditions such as age-related macular degeneration, glaucoma, diabetic retinopathy and other types of retinal and optic nerve head disease.

The feature extractor 202/204, for example as illustrated in FIG. 5, may start with a 3D convolution layer, followed by batch normalization and ReLU activation. For example, in the reconstruction branch 112, embodiments of the present disclosure may start with volumetric adjacent slices. Therefore, the 3D convolution layer can then be used to encode the feature prior to the DRM 202/212 to reduce the dimensionality of the features from 3D to 2D. The upsampler 206/208, which is used for transforming the first reduced-dimension feature data generated by the reconstruction downsampler 202/204 to the first upsampled data, may start with a 2D convolutional layer, followed by batch normalization and ReLU activation. The feature extractor 214/216 used for the segmentation branch 108 may contain a 2D convolutional layer, followed by batch normalization and ReLU activation. The segmentation upsampler 218/220 for transforming the first reduced-dimension feature data generated by the segmentation downsampler 214/216 to first upsampled data having the same dimensions may start with a 2D convolutional layer, followed by batch normalization and ReLU activation.

The proposed SA-Net for volumetric segmentation of the choroid was evaluated. The choroid is clinically of interest as the vascular layer of the eye, providing upwards of 60% of the blood supply to the retina. Variations in the choroid have been linked to many ocular conditions, including age-related macular degeneration and diabetic retinopathy. Until recently, OCT imaging of the choroid has been challenging, as it is obscured by the highly scattering retinal pigment epithelium and visibility of the choroid was highly limited using spectral domain OCT systems operating at the 800 nm range. However, the adoption of swept-source lasers operating at 1000 nm into OCT systems has provided a window of opportunity for choroidal analysis due to reduced scattering.

The proposed SA-Net was evaluated on two OCT datasets. The first data set is composed of 40 high myopia eyes acquired using a commercial swept-source OCT (SS-OCT) system, DRI OCT Triton (Topcon Corp., Japan) with a 1050 nm wavelength, scanning speed of 100,000 A-scans/sec and 7 mm × 7 mm scanning protocol, centred at the macula. Each eye volume in the Triton data set contains 256 slices with dimensions 256 × 128. Another separate data set is obtained by acquiring scans from nine normal eyes using the PLEX Elite 9000 SS-OCT system (Carl Zeiss Meditec, Jena, Germany) operating at a wavelength range between 1040 nm and 1060 nm, with a scanning speed of 100,000 A-scans/sec and 15 mm × 9 mm scanning protocol. Each eye volume in the PLEX data set contains 834 slices with dimension 512 × 500. Prior pre-processing is performed to limit the field of view of the acquired scans to the macula region and to resize the dimensions to 256 × 128. The network receives the target slice for segmentation together with the adjacent slices as inputs for reconstruction. Slices from the ends of the volume are padded by averaging the target slice with the available adjacent slices.

The segmentation result was evaluated volumetrically by calculating the IoU, dice score and accuracy over a volume, with respect to ground truth segmentation. The inter-slice correlation was assessed by measuring the quality of the choroidal thickness map generated from the choroidal segmentation. The method was repeated for a plurality of target slices, and further comprised generating a choroidal thickness map from segmentation of the plurality of target slices. In particular, the choroidal thickness map was obtained by stacking the choroidal thickness obtained from each slice. The generated map was evaluated by calculating the structural similarity index, which assesses the similarity of the predicted thickness map and ground truth thickness map. Given two images with the same dimension, x and y, SSIM formula is given by

$$S S I M = \frac{\left(2 {μ}_{x} {μ}_{y} + {c}_{1}\right) \left(2 {σ}_{x y} + {c}_{2}\right)}{\left({μ}_{x}^{2} + {μ}_{y}^{2} + {c}_{1}\right) \left({σ}_{x}^{2} + {σ}_{y}^{2} + {c}_{2}\right)} ,$$

where µₓ, µ_{y}, σₓ, σ_{y}, σ_{xy} are the average of x, the average of y, the variance of x, the variance of y, and the covariance of x and y respectively. While c₁ = (0.001DR)² and c₂ = (0.003DR)². In the present disclosure, DR or dynamic range is given by:

$$D R = m a x \left({y}_{\text{thicknessmap}}^{\text{true}}\right) - m i n \left({y}_{\text{thicknessmap}}^{\text{true}}\right) .$$

Table 1 shows the result comparison for the Triton data set using the proposed SA-Net with other segmentation approaches such as 3D U-Net, BC U-Net and GGPF-Net. The results show that the SA-Net architecture has successfully outperformed other architectures for volumetric segmentation. This demonstrates that learning the adjacent spatial features explicitly from reconstruction enabled more precise 3D volumetric segmentation.

TABLE 1

| Method | IoU | Dice | Acc | SSIM |
|---|---|---|---|---|
| 2D U-Net | 0.9144 | 0.9551 | 0.9923 | 0.6047 |
| 3D U-Net | 0.9011 | 0.9477 | 0.9911 | 0.5798 |
| BC U-Net | 0.9204 | 0.9583 | 0.9929 | 0.6344 |
| GGPF-Net | 0.9166 | 0.9560 | 0.9926 | 0.6362 |
| SA-Net | 0.9221 | 0.9592 | 0.9930 | 0.6379 |

Table 2 also shows the result for the PLEX data, where the proposed architecture achieved similar results. It is also important to take note that the network complexity and computational power needed for the present architecture are much less than those needed for BC U-Net, resulting in faster learning and inference time.

TABLE 2

| Method | IoU | Dice | Acc | SSIM |
|---|---|---|---|---|
| 2D U-Net | 0.7793 | 0.8744 | 0.9793 | 0.3028 |
| 3D U-Net | 0.7836 | 0.8767 | 0.9802 | 0.3149 |
| BC U-Net | 0. 7988 | 0.8865 | 0.9817 | 0.3126 |
| GGPF-Net | 0.7560 | 0.8585 | 0.9773 | 0.3005 |
| SA-Net | 0.7927 | 0.8829 | 0.9811 | 0.3091 |

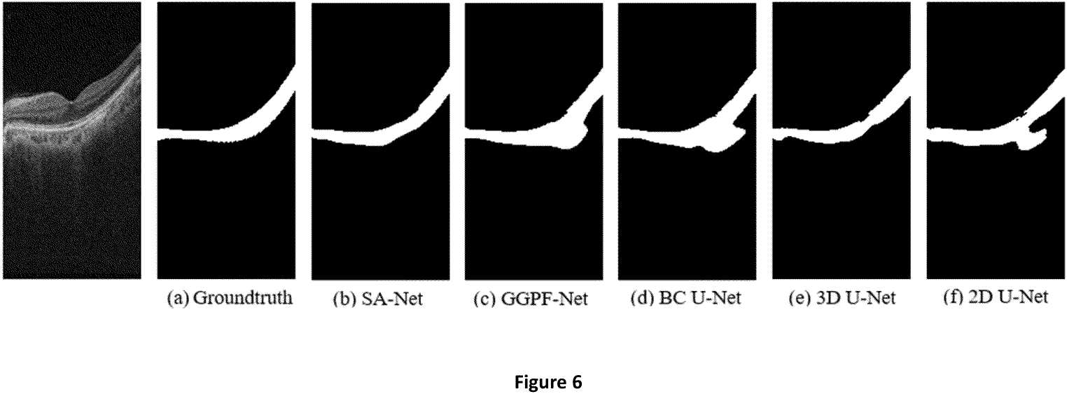

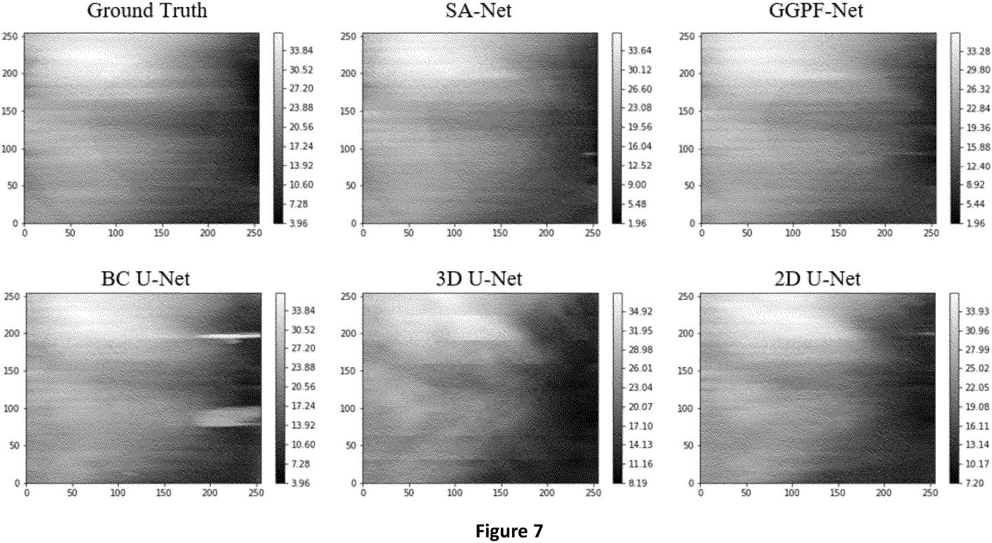

FIG. 6 shows, from left to right, the original image, the ground truth (FIG. 6(a)), and the 2D segmentation result for each architecture (FIGS. 6(b) to 6(f)), indicating the segmentation result achieved by the respective architectures. It can be seen that SA-Net and 3D U-Net generate the best (i.e. closest to ground truth) segmentations. FIG. 7 shows the choroidal thickness map generated from the ground truth and from each of the segmentation architectures. It is observed that the thickness map from the 2D-based U-Net is noisier with more variability between slices, while the block-based 3D U-Net resulted in a more patchy result. BC U-Net, GGPF-Net, and SA-Net showed results which were more similar to the ground truth. SA-Net is shown to perform better than the other approaches in both data sets for the SSIM score as shown in Table 1 and Table 2, and visually compared to the ground truth choroidal thickness map.

Table 3 shows a detailed comparison between the proposed SA-Net and the state-of-art networks. Spatial information can provide useful context for volumetric segmentation. In the proposed SA-Net, incorporating spatial information from corresponding adjacent slices enabled our proposed SA-Net architecture to explicitly integrate spatial correspondences. SA-Net was compared with other recent approaches to segment the choroid in volumetric OCT images from two different commercial devices, and it was demonstrated that SA-Net outperformed the other approaches in segmentation accuracy and quality of the generated choroidal thickness map, with lesser computational requirements. The results show that SA-Net could be used for efficient and accurate segmentation of OCT data as well as potentially other volumetric medical images.

TABLE 3

| | | | | | | |
|---|---|---|---|---|---|---|
| | SA-Net | 2D U-Net | 3D U-Net | BC U-Net | GGPF-Net | Bio-Net |
| Automation | Full | Full | Full | Full | Full | No |
| Computational Resources | Light | Light | Heavy | Heavy | Light | Light |
| Robustness to Noise | Good | Poor | Poor | Good | Moderate | Moderate |

Also disclosed herein is a non-transitory computer-readable storage having instructions stored thereon for causing at least one processor to carry out the disclosed method.

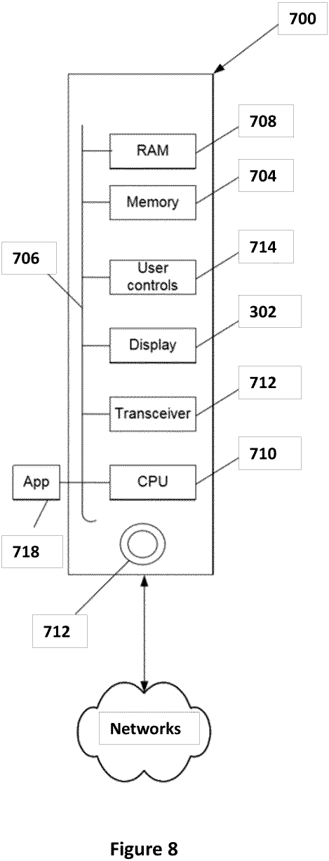

FIG. 8 is a block diagram showing an exemplary computer device 700, in which embodiments may be practiced. The computer device 700 may be a mobile computer device such as a smart phone, a wearable device, a palm-top computer, and multimedia Internet enabled cellular telephones, an on-board computing system or any other computing system, a mobile device such as an iPhone™ manufactured by Apple™, Inc or one manufactured by LG™, HTC™ and Samsung™, for example, or other device.

As shown, the mobile computer device 700 includes the following components in electronic communication via a bus 706:
(a) a display 302;
(b) non-volatile (non-transitory) memory 704;
(c) random access memory (“RAM”) 708;
(d) N processing components 710;
(e) a transceiver component 712 that includes N transceivers; and
(f) user controls 714.

Although the components depicted in FIG. 8 represent physical components, FIG. 8 is not intended to be a hardware diagram. Thus, many of the components depicted in FIG. 8 may be realized by common constructs or distributed among additional physical components. Moreover, it is certainly contemplated that other existing and yet-to-be developed physical components and architectures may be utilized to implement the functional components described with reference to FIG. 8.

The display 302 generally operates to provide a presentation of content to a user, and may be realized by any of a variety of displays (e.g., CRT, LCD, HDMI, micro-projector and OLED displays).

In general, the non-volatile data storage 704 (also referred to as non-volatile memory) functions to store (e.g., persistently store) data and executable code. The system architecture may be implemented in memory 704, or by instructions stored in memory 704.

In some embodiments for example, the non-volatile memory 704 includes bootloader code, modem software, operating system code, file system code, and code to facilitate the implementation components, well known to those of ordinary skill in the art, which are not depicted nor described for simplicity.

In many implementations, the non-volatile memory 704 is realized by flash memory (e.g., NAND or ONENAND memory), but it is certainly contemplated that other memory types may be utilized as well. Although it may be possible to execute the code from the non-volatile memory 704, the executable code in the non-volatile memory 804 is typically loaded into RAM 708 and executed by one or more of the N processing components 710.

The N processing components 710 in connection with RAM 708 generally operate to execute the instructions stored in non-volatile memory 704. As one of ordinarily skill in the art will appreciate, the N processing components 710 may include a video processor, modem processor, DSP, graphics processing unit (GPU), and other processing components.

The transceiver component 712 includes N transceiver chains, which may be used for communicating with external devices via wireless networks. Each of the N transceiver chains may represent a transceiver associated with a particular communication scheme. For example, each transceiver may correspond to protocols that are specific to local area networks, cellular networks (e.g., a CDMA network, a GPRS network, a UMTS networks), and other types of communication networks.

It should be recognized that FIG. 8 is merely exemplary and in one or more exemplary embodiments, the functions described herein may be implemented in hardware, software, firmware, or any combination thereof. If implemented in software, the functions may be stored on or transmitted over as one or more instructions or code encoded on a non-transitory computer-readable medium 704. Non-transitory computer-readable medium 704 includes both computer storage medium and communication medium including any medium that facilitates transfer of a computer program from one place to another. A storage medium may be any available medium that can be accessed by a computer.

It will be appreciated that embodiments of the present disclosure provides a novel segmentation architecture that is capable of fully automated three-dimensional segmentation of volumetric medical image data. This architecture encompasses the following key novel aspects. First, soft parameter sharing aggregates the spatial features more explicitly by directly learning the correlation between adjacent slices and the slice that will be segmented. In addition, simultaneous reconstruction and segmentation extracts intra-slice features which are directly used for segmentation. Further, automated generation of volumetric choroidal representation enables 3D visualization of the choroid. Last but not least, generation of full-field choroidal thickness maps enables enface analysis of thickness variations in the choroid across the retina.

It will be appreciated that many further modifications and permutations of various aspects of the described embodiments are possible. Accordingly, the described aspects are intended to embrace all such alterations, modifications, and variations that fall within the spirit and scope of the appended claims.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

1-17. (canceled)

18. A method of segmenting a volumetric image comprising a plurality of slices, the method comprising:
inputting a target slice of the volumetric image to a deep neural network (DNN) having a multi-task learning architecture, the multi-task learning architecture comprising:
a segmentation DNN that is configured to output a segmentation of the target slice; and
a reconstruction DNN that is configured to:
receive a plurality of adjacent slices to the target slice; and
output a reconstruction of the target slice based on the plurality of adjacent slices;
wherein the reconstruction DNN is further configured to share spatial information with the segmentation DNN, the spatial information being indicative of correlations between the adjacent slices and the target slice.

19. A method according to claim 18, wherein the reconstruction DNN comprises a convolutional feature extractor for generating first feature data from the adjacent slices, and a reconstruction downsampler for generating first reduced-dimension feature data from the first feature data at one or more scales.

20. A method according to claim 19, wherein the reconstruction DNN comprises a reconstruction upsampler for transforming the first reduced-dimension feature data to first upsampled data having the same dimensions as the first feature data.

21. A method according to claim 19, wherein the reconstruction DNN comprises one or more dimension reduction layers for applying a dimension reduction mechanism to the first feature data and/or to the first reduced-dimension feature data.

22. A method according to claim 21, wherein the dimension reduction mechanism comprises:
inputting the first feature data and/or the first reduced-dimension feature data to a 3D convolution layer;
applying an aggregation of features between adjacent slices;
applying batch normalization to the output of the 3D convolution layer; and
applying a ReLU activation function to the output of the batch normalization.

23. A method according to claim 21, wherein layers of the reconstruction downsampler are connected to layers of the reconstruction upsampler via respective ones of the dimension reduction layers by concatenation.

24. A method according to claim 18, wherein the segmentation DNN comprises a convolutional feature extractor for generating second feature data from the target slice, and a segmentation downsampler for generating second reduced-dimension feature data from the second feature data at one or more scales.

25. A method according to claim 24, wherein the segmentation DNN comprises a segmentation upsampler for transforming the second reduced-dimension feature data to second upsampled data having the same dimensions as the second feature data.

26. A method according to claim 24, wherein layers of the segmentation downsampler are connected to layers of the segmentation upsampler.

27. A method according to claim 24, wherein the reconstruction DNN is configured to share spatial information with the segmentation DNN by element-wise addition of output of layers of the reconstruction upsampler to output of layers of the segmentation upsampler.

28. A method according to claim 18, wherein the loss function of the segmentation DNN is the 2D Intersection over Union (IoU) loss function.

29. A method according to claim 18, wherein the volumetric image is a 3D medical image.

30. A method according to claim 29, wherein the 3D medical image is a 3D optical coherence tomography (OCT) image.

31. A method according to claim 30, wherein the 3D OCT image is a retinal image, and wherein the target slice corresponds to a layer of the choroid.

32. A method according to claim 31, wherein the method is repeated for a plurality of target slices, and wherein the method further comprises generating a choroidal thickness map from segmentation of the plurality of target slices.

33. A system for segmentation of a volumetric image comprising a plurality of slices, comprising:
at least one processor; and
computer-readable storage having stored thereon instructions for causing the at least one processor to carry out a method according to claim 18.

34. Non-transitory computer-readable storage having instructions stored thereon for causing at least one processor to carry out a method according to claim 18.
